(19) United States
(12) Patent Application Publication
(10) Pub. No.: US 2008/0153125 A1
(43) Pub. Date: Jun. 26, 2008

(54) RAPID DETECTION OF MICROORGANISMS IN FLUIDS

(75) Inventors: Daniel A. BUTTRY, Laramie, WY (US); Angela M. OPPEDAHL, Boone, IA (US); Robert CZARNEK, Johnstown, PA (US); Martin C. FOSTER, Nevada, IA (US); Steven J. LASKY, Ankeny, IA (US); Ho-Ming PANG, Ames, IA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: ADVANCED ANALYTICAL TECHNOLOGIES, INC., Ames, IA (US)

(21) Appl. No.: 11/937,992

(22) Filed: Nov. 9, 2007

Related U.S. Application Data
(60) Provisional application No. 60/858,212, filed on Nov. 10, 2006.

(51) Int. Cl. C12Q 1/24; C12M 1/34; C12M 1/00

(52) U.S. Cl. 435/30; 435/287.1; 435/287.9; 435/308.1

(57) ABSTRACT

A system for the rapid detection of microbial contamination in a fluid sample such as water, involving the use of a filter material having a surface adapted to receive the sample in order to retain substantially all microbes from the sample on the filter surface under conditions that minimize the potential for contamination from sources other than the sample itself, and in a manner that permits the filter surface to be incubated in order to grow viable microbes contained thereon, in combination with a growth medium and an analytic instrument to permit analysis of the filter surface, within a predetermined incubation period, in order to determine whether the growth onset of viable microbes that may be present on the surface has begun.

### RELATED APPLICATION

This application claims priority to U.S. provisional application Ser. No. 60/858,212, which was filed on Nov. 10, 2006, and which is hereby incorporated by reference, in its entirety.

### TECHNICAL FIELD

This invention relates to the detection of bacteria, yeast, mold and other microorganisms in fluids such as high purity water such as drinking water (DW), purified water (PW) and water for injection (WFI). It specifically relates to the detection of viable microorganisms in such water samples.

### BACKGROUND OF THE INVENTION

Various instruments and methods exist for use in testing water and other liquids for microbial contamination, including those that rely on the use of filtering a sample of the water, which is then incubated with a suitable media in order to grow and detect any microbes that may have been present. See, for instance, Millipore Technical Publications “Microfil V Filtration Device”.

Over the years, there has been a particular interest in the rapid detection of microbes in such samples.

Various methods can be used to provide corresponding outcomes, as between detection, identification, enumeration of the microbes that may be present. In turn, these methods can be based on a wide variety of principles and approaches, including those relating to biochemical activity, DNA content, antibody binding, and so on. Each method carries its corresponding benefits and drawbacks, including with respect to the ability to distinguish between living and non-living cells, between viable and non-viable cells, between cells and particulate matter, and so forth.

On a different subject, filters have been developed to achieve a wide variety of purposes, including many types that are designed for filtering microbes from water and other such samples. Filters have been made in many and various shapes and sizes, from many materials, and having various physical chemical structures and characteristics.

On yet another subject, various detection mechanisms, and corresponding instrumentation, exist for use in an equally wide array of purposes. Such mechanisms can include, for instance, the use of optical, mechanical, biochemical and other properties.

One such mechanism involves the use of optical microscopy, including what is known as “darkfield” microscopy, which involves an optical microscopy illumination technique used to enhance the contrast in unstained samples. It works on the principle of illuminating the sample with light that will not be collected by the objective lens, so not form part of the image. This produces the classic appearance of a dark, almost black, background with bright objects on it.

In spite of these various capabilities and interests, to this day, Applicants are not aware of any method or corresponding system that can be used to detect the presence of microbes in liquid samples, and particularly viable microbes, in a manner that provides an optimal balance of speed, cost, minimal risk of contamination, and applicability to potentially very low levels of microbes in fluid samples such as water. In turn, the water industry continues to rely, in large part, on conventional plating and incubation methods that have been in existence for decades or more, and that require on the order of a day or more to generate detectable results, generally in the form of visible colony formation.

### BRIEF DESCRIPTION OF THE DRAWING

In the Figures,

FIG. 1 shows a microfabricated filter design in which pores run perpendicularly through the filter, and where the regions containing such pores are supported by more mechanically stable, thicker regions of the filter material.

FIG. 2 shows a microfabricated filter design in which the flow path runs into and then laterally though the filter, as shown by the arrow in the figure.

FIG. 3 shows another lateral flow design in which the layout of the flow paths is differently arranged than in FIG. 2.

FIG. 4 shows the surface of an aluminum oxide filter produced by anodization of an aluminum surface to grow an oxide layer with the geometry in the figure, followed by removal of the oxide layer comprising the filter using solvent etching or other processing steps.

FIG. 5 shows the top surface and a cross-sectional view of a polycarbonate track-etched filter in which pores are produces by the passage of high energy particles through the filter, followed by chemical development of the tracks to produce pores that run perpendicularly through the filter.

FIG. 6 shows a track etched mica filter with square pores that run perpendicularly through the filter.

FIG. 7 shows a close-up of the top surface and a cross-sectional view of a track etched mica filter.

FIG. 8 shows a schematic view of a darkfield illuminator.

FIG. 9 shows a picture of an experimental apparatus comprising a darkfield illuminator, a sample stage holding the sample (a filter), a collection optic (a microscope) and a detection device (a CCD camera).

FIG. 10 shows a false color image of the surface of a gold-coated polycarbonate membrane onto which bacterial cells of Bacillus cereus have been captured by filtration.

FIG. 11 shows the same filter surface as in FIG. 10 after the filter was placed on a growth medium for two hours, allowing the cells the opportunity to grow.

FIG. 12 shows the same filter surface as in FIG. 10 after the filter was placed on a growth medium for three hours, allowing the cells the opportunity to grow.

FIG. 13 shows an image of a gold-coated polycarbonate filter onto which bacterial cells of E. coli have been captured by filtration.

FIG. 14 shows the same filter surface as in FIG. 13 after the filter was placed on a growth medium for 50 minutes, allowing the cells the opportunity to grow.

FIG. 15 shows the same filter surface as in FIG. 13 after the filter was placed on a growth medium for two hours, allowing the cells the opportunity to grow.

FIG. 16 shows the manner in which microfabrication techniques may be used to produce a filter with perpendicular pores that pass through the filter.

### SUMMARY OF THE INVENTION

The present invention provides a system for the rapid detection of microbial contamination in a fluid sample such as water, the system comprising:

a) a filter assembly comprising a filter material comprising a surface adapted to receive the sample in order to retain microbes from the sample, and preferably substantially all microbes, on the filter surface under conditions that minimize the potential for contamination from sources other than the sample itself, and in a manner that permits the filter surface to itself then be incubated in order to permit the growth onset of viable microbes contained thereon;

b) a growth medium adapted to permit the incubation and growth of microbes that may be retained on the filter surface, and

c) an analytic instrument adapted to permit analysis of the filter surface, within a predetermined incubation period, in order to determine whether and/or the extent to which the growth onset of viable microbes that may be present on the surface has begun; whereby, the system can provide either qualitative and/or quantitative results regarding the onset of microbial growth, and in turn, can be used to determine and/or distinguish as between the existence of a) particulate matter in the sample, b) living cells that are not culturable under the conditions of use, and/or c) cells that are both living and culturable, as evidenced by their ability to exhibit the onset of detectable growth on the surface within the predetermined incubation period. Typically, the system will be used to distinguish living, culturable cells from any other materials (e.g., particulate matter together with non-culturable cells) that may be present in the sample. In turn, the present invention provides various combinations and subcombinations of components, which have the potential to be novel in their own right, including filter materials and/or growth media adapted for use in a method as described herein.

In a preferred embodiment, the system can be used to rapidly detect or determine cell presence, in that the predetermined incubation period is considerably shorter than corresponding conventional culture periods, e.g., the period can be on the order of eight hours or less, preferably six hours or less, and more preferably four hours or less. This can be compared to on the order of a day or more using conventional, plating and growth, techniques. Unless otherwise indicated, the word “cell(s)” as used herein shall refer to microbial cells, including bacterial, yeast and mold cells, to be detected and/or determining in the sample described.

In turn, a preferred sample of this invention can be selected from gaseous, vapor, and liquid, and is preferably liquid, and more preferably water. Preferably a sample protocol is used to obtain a sample from a liquid that is expected to have, at most, a very low level of microbial contamination, the protocol comprising the use of aseptic sampling.

In one preferred embodiment, the filter assembly includes a holder (e.g., frame) within or upon which the filter may be supported, and often further includes a tapered housing that serves to direct the fluid flow through the filter, and a cap to maintain sterility of the filter prior to and after use. The filter assembly may also include a cap that can contain growth medium, so that the filter may be placed directly into the cap, and thereby exposed to the growth medium, thereby affording any cells that may be present the opportunity to grow.

In turn, the filter material is preferably provided in the form of a membrane, and in turn, can be provided as a wafer, sheet, or other suitable shape or type. The material itself can be selected from the group consisting of polycarbonate, polyimide and other polymers that may be used in the track etch process; aluminum oxide (alumina); silicon, silicon dioxide, epoxy, photoresist and other materials that may used in the microfabrication process; various types of glass, such as fused silica, borosilicate glass, etc., that may be formed into capillaries and fused together, cut into sheets and polished to form filters with pores that run perpendicularly though the filter. Examples of suitable filter materials include, but are not limited to, Si, Al oxide, cellophane, etc.

In a preferred embodiment, the filter material provides a filter surface that is adapted (e.g., by physical/chemical characteristics that include shape, size, porosity, surface and the like) to retain any microbes that may be present in the sample, on the surface of the filter itself, such that they can be grown and detected using microscopy. In a particularly preferred embodiment, the microscopy is darkfield microscopy, or any other microscopic technique suitable to detect the presence of a relatively small number of growing cells upon a substantially flat surface. See, for example, Nikon's tutorial at http://www.microscopyu.com/articles/stereomicroscopy/stereodarkfield.html, the entire disclosure of which is incorporated by reference, where it provides that darkfield observation in stereomicroscopy requires a specialized stand containing a reflection mirror and light-shielding plate to direct an inverted hollow cone of illumination towards the specimen at oblique angles. The principal elements of darkfield illumination are the same for both stereomicroscopes and more conventional compound microscopes, which often are equipped with complex multi-lens condenser systems or condensers having specialized internal mirrors containing reflecting surfaces oriented at specific geometries.

The filter material also provides the ability to filter a suitable, preferably predetermined amount of the liquid sample, in a manner that provides a desired and suitable combination of flow rate, lack of fouling, and other performance characteristics. Applicants have discovered, inter alia, that membranes can be found or prepared given the present description, in a manner that provides each of these preferred capabilities, namely, the ability to retain cells on, rather than below, the filter surface, and then permit the growth of the retained cells substantially in situ on the surface, while also providing suitable flow and other characteristics for their intended use.

A preferred filter material provides an optimal combination of such properties as chemical, optical, fluidic flow properties. It is particularly preferred that the filter provide an optically flat surface so as to allow the darkfield imaging to identify cell growth for any and all cells on the filter surface.

In a preferred embodiment, the analytic instrument is selected from the group consisting of a microscope (e.g., darkfield or phase contrast), a spectrophotometer, and a spectrophotometer that has imaging capabilities, such as an infrared microscope. In a particularly preferred embodiment, the instrument is a microscope and further comprises a darkfield microscope.

Applicability of the invention is not limited to DW, PW and WFI, as there are other types and designations of water that also may benefit from the analyses described below. Similarly, the techniques described here also may be used to detect microbes in other liquids, such as beverages, contact lens fluid, recreational water (such as lake water) and other liquids for which low levels of microorganisms are desirable. It also may be used to detect airborne microorganisms, such as in clean rooms, hospitals and other facilities where airborne microorganisms may cause undesirable effects. In such cases, the microorganisms may first be captured by passage of a quantity of air through a filter such as those described below or by passage through a quantity of pure water into which the microorganisms become trapped, followed by treatment of that water sample just as described below for other types of water samples. For simplicity, the discussion below is largely limited to water samples.

Typical maximum allowable counts of microorganisms in water are 500 colony forming units per mL (cfu/mL) for DW, 100 cfu/mL for PW and 10 cfu per 100 mL for WFI. These sample matrices have different characteristics. For example, drinking water often has relatively high levels of particulates, unlike purified water and WFI. Further, drinking water may have substantial numbers of dead microorganisms, while PW and WFI should not have substantial numbers of dead microorganisms or fragments of such dead cells. There also should be no (or very low levels of) particulates in PW and WFI, because these two types of water are often filtered and/or have been passed through reverse osmosis membranes and/or have been distilled. Thus, the detection of microorganisms in PW and WFI is aided by the fact that there should be no intact or fragmented cells of microorganisms of any type, dead or alive, nor should there be high numbers of particulates.

The present invention further provides filter assemblies, filter materials, and analytic instruments suitable for use with a system of the present invention, several embodiments of which can be considered novel in their own right, together with methods of preparing and methods of using each.

A filter assembly of this invention can include a filter material suitable to filter the sample in order to retain microbes present therein, to then retain the filter material in the course of incubation and growth of microbes that may be retained on the filter surface, and to then be operably coupled with the analytic instrument to permit analysis of the filter surface. A filter material of this invention can inlcude a filter material that has been microfabricated to permit the flow of a sample therethrough, in a manner that permits substantially all microbes contained in the sample to be retained on the surface thereof. An analytic instrument (e.g., darkfield microscope) of this invention can include one that has been adapted to retain the filter assembly and/or filter material, in the course of analyzing the filter surface in order to determine whether and/or the extent to which the growth onset of viable microbes that may be present on the surface has begun.

### DETAILED DESCRIPTION

Applicants have discovered, recognizing at least in part that PW and WFI are fairly pure matrices that do not contain substantial numbers of intact cells of microorganisms, fragments of cells of microorganisms or particulates, that it is possible to detect microorganisms using a method as described herein.

Fluid (e.g., water) samples can be obtained using any of several possible sampling protocols, and preferably by the use of a filter membrane adapted to remove and retain any microbes that may be present. The membrane, in turn, provides desired characteristics, especially with regard to its chemical, optical and fluidic flow properties.

The surface of the membrane can be analyzed for the presence of microbes using any of a variety of optical and/or spectroscopic methods, some of which may involve obtaining images of the surface. Such methods can be enhanced by virtue of the chemical and/or optical characteristics of the membranes described herein, thereby providing unique combinations of capture and optical inspection that enable the detection of microorganisms in a reasonable time frame, thereby providing advantages over other previously known approaches. The analysis described herein can involve directly observing growth of the cells on the filter surface used for their capture. This unambiguously demonstrates viability of the microorganisms.

Those skilled in the art, given the present description, will appreciate the manner in which a substantially similar set of protocols can be used to capture and detect airborne microorganisms, for instance, from the air in clean rooms, hospitals, etc. In such cases, the protocol will generally involve filtering larger volumes of air using filters much like those described below for water and other aqueous samples, with appropriate modifications for high volume air sampling. Detection can be done as described herein, or in various other ways that will become apparent to those skilled in the art, given the present description.

One aspect of the present invention involves sampling from a fluid such as drinking water, purified water or WFI systems in a sterile manner. The benefits of sterile sampling derive predominantly from the lack of false positives. For example, at the present time samples are drawn from WFI systems using a nearly sterile protocol that may involve workers wearing sterile clothes, gloves, hats to cover their heads, and masks to cover the nose/mouth area as well as any facial hair. These samples are then filtered, and the filters are placed on culture media and cultured for some period of time, often as long as 14 days. In spite of precautions to maintain sterility, WFI testing for bacterial contamination sometimes results in positives that come from bacteria that derive from the workers themselves. An example of such an organism might be any of a variety of Staphylococcus organisms, such as Staphylococcus aureus, which often colonize the nasal cavity of humans. The presence of such an organism in a WFI bacterial test almost invariably occurs because some action of the worker led to contamination of the sample. Thus, it is important that sampling be done in as sterile an environment as possible. Toward this end, it is preferred that the sampling protocol and sampling apparatus be as sterile as possible, employing aseptic technique (such as gowns and gloves, cleaning the spigots or letting initial fluid run through before sampling), thereby eliminating nearly all unwanted false positives that derive from microorganisms arising from environmental sources.

One preferred approach to sampling WFI and other water samples in this disclosure is to use a filter holder that is compatible with the various types of vacuum filtration manifolds available to the user. Currently, many vacuum manifolds accept multiple filter holders. The filter holder typically has an upper chamber where the water sample is added, a removable cap that covers this chamber, a tapered region that shapes the flowing stream so that it matches the size of the active area of the filter material at the bottom, and a detachable fixture at the bottom that may hold the filter itself. The cap may also be used to hold a growth medium that the filter may be placed onto if it is desirable to culture any microorganisms that may be captured on the filter after the filtration. In this case, the detachable fixture holding the filter would be designed such that it can be placed onto the cap so that the filter itself may be in contact with the growth medium. The material from which the filter holder may be fabricated includes a wide variety of polymeric materials, such as polyethylene, polycarbonate and the like, as well as glass or metal.

The sampling fixtures used for sampling of gasses tend to be somewhat different than the vacuum filtration manifolds described above. Air sampling is typically also done by using a vacuum or air pump to draw or push air though a filter, respectively. Several types of devices are known to those skilled in the art of air sampling via filtration.

The purpose of the filter holder is to accept and filter the water sample from the water system. Thus, the sample container contains a means for filtering said water sample. The characteristics of a preferred filter will be described more fully herein. Its purpose is to capture on its surface any bacteria that may have been present in the water sample. Thus, in a typical sampling protocol, water may be transferred from the water system into the upper chamber of the filter holder and filtered through the filter. Following filtration, the filter is then transferred under sterile conditions into an inspection system that will be described more fully below.

For air sampling, the filter holder may be of different design than those relevant for water sampling. This is because it need not hold the water sample prior to its filtration. Instead, it may only hold the filter while air is drawn or pushed through it.

A preferred procedure for using the filter holder is described below, though those skilled in the art will find other approaches and procedures suitable as well, given the present specification. First, the filter holder is removed from its sterile packaging. Then, it is connected to a vacuum filtration manifold and the cap is removed. Then, the water sample is added into the top chamber in the filter holder. Then, the vacuum is applied. This draws the water through the filter, thereby effecting capture of microorganisms, particles and other insoluble debris on the surface of the filter. Then, the detachable funnel connected to the filter is disconnected from the filter holder so that the fixture can be placed into an optical inspection station that will be more fully described below. The descriptions here comprise only a few examples of the ways in which the filter holder may be configured and used. These descriptions should not be interpreted as limiting.

The present invention provides a system for the rapid detection of microbial contamination in a fluid sample such as water, the system comprising a filter assembly comprising a filter material comprising a surface adapted to receive the sample in order to retain substantially all microbes from the sample on the filter surface under conditions that minimize the potential for contamination from sources other than the sample itself.

Preferred membranes for cell capture have optical properties that allow for the observation and detection of bacterial cells captured at the membrane surface. Thus, these membranes should have substantially smooth surfaces. In a preferred embodiment, they are optically smooth, i.e. with root-mean-square surface roughness less than ¼ of the wavelength of light used for the optical and/or spectroscopic detection methods. A variety of methods are available for preparing such membranes. In another embodiment, these membranes are atomically flat. An example of such an atomically flat membrane is the surface of a sheet of mica (either natural or synthetic) that has holes through it such that the mica can act as a filtration membrane.

Another example of a membrane that has regions that are nearly atomically flat is a Si single crystal wafer, such as is used in integrated circuit microfabrication. In another embodiment, the membranes are formed on silicon wafers such as those used in integrated circuit microfabrication. In this embodiment, the membranes are comprised of Si wafers with open structures created on their surface and/or through the body of the wafer such that flow can occur through these open structures.

In one embodiment, the membranes are optically flat and comprised of a solid material having open structures that can be comprised of pores that run entirely through the thickness of the material. In another embodiment, these open structures can be lateral openings, such that flow occurs in the plane of the surface of the membrane, as described more fully below. In such a case, fluid could be removed laterally from the wafer, such that fluid would enter the wafer at some region and be removed at a place that is laterally displaced from the entry point. In this case, fluid may not need to be removed through the back side of the wafer. These lateral openings also may be connected with other openings that allow fluid to pass through the wafer, emerging from the back side. In such a case, fluid may be removed from the back side of the wafer.

In another embodiment the membranes are optically transparent. This characteristic can be used to advantage in the detection, for example by providing the ability to observe cells on the membrane surface either from the front side or the back side (i.e. looking through the membrane). It also makes it possible to pass light through the membrane material and/or to use the membrane as an optical waveguide. This property will be described further below as it relates to one approach for cell detection. In all of these cases, an important characteristic of a particularly preferred membrane surface is that it be substantially free of optical imperfections, especially those that have sizes and shapes similar to the cells of microorganisms. The term “substantially free” as used in this regard, refers to a surface that is sufficient free of imperfections to the point where it can be used for the purpose of the present invention.

Examples of the types of membranes that can be used for capturing microorganisms include, but are not limited to, filters microfabricated from silicon, aluminum oxide membranes, cellophane or cellulose-based membranes, polycarbonate membranes, membranes formed from other polymeric materials, membranes formed from combinations of materials (such as a combination of silicon, silicon dioxide and/or other polymers), and mica membranes. In such cases, the membranes may have perpendicular pores which may be circular or may also have other shapes. Optionally, they can have flow paths with shapes other than circular (such as a long narrow gap through which filtration may take place) of a sufficiently small dimension so that the targeted cells can be captured on the membrane surface. In other words, the flow paths must be small enough so that the cells cannot pass through them, and thereby are caused to remain on the surface of the membrane.

These flow paths can be made in a variety of ways including, but not limited to, using photolithographic methods such as those used in the semiconductor industry including patterning with photoresist and using various chemical etching steps, reactive ion etching, track etching (i.e. using radioactive particles to make tracks through the membranes that can later be etched using chemical etching solutions), chemical etching without prior tracking, punching holes mechanically, ablating holes using various wavelengths of light, and forming the membrane in a mold that has posts in it or on a surface that has posts on it so the pores are formed at the time the membrane is first formed. It is important that the pores not be larger than the size of the cells to be captured. The standard pore size used for filtration at this time in the industry is 0.45 micrometers. However, because of the distribution of pore sizes resulting from many of the processes used to make filters, the mean or median pore sizes for such filters may deviate substantially from this size. Further, it may be desirable to use smaller pore sizes so as to capture even smaller microorganisms. Thus, in a preferred embodiment, the filtration membranes may have pore sizes or flow path sizes ranging from approximately twenty nanometers up to approximately five micrometers. In a preferred embodiment, the pore size or flow path size may be between about 0.1 microns and about 1 micron, and more preferably, between about 0.2 microns and about 0.45 microns, inclusive.

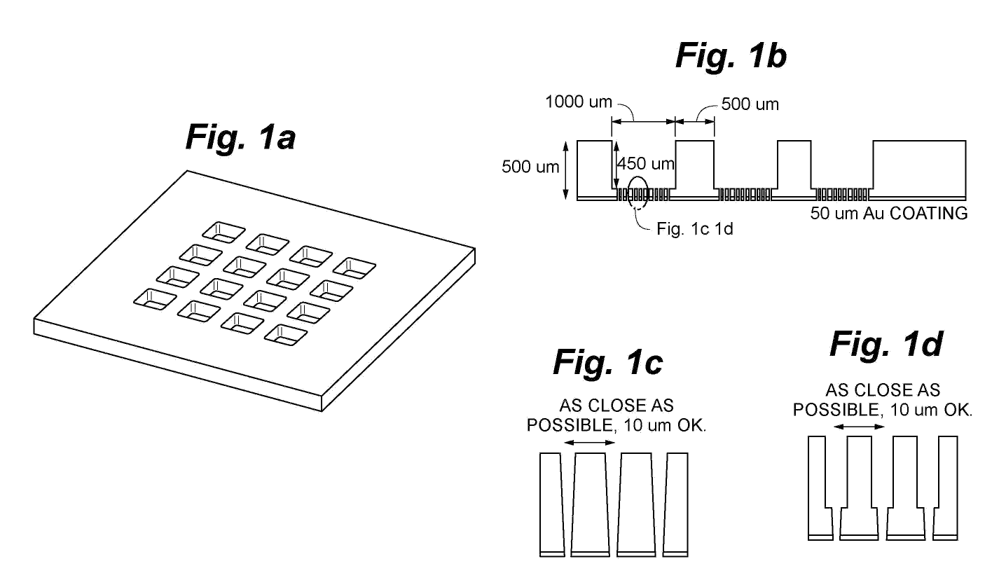

The images shown in the figures provide examples of some of the materials and types of flow paths that can be used for filtration according to the present invention. FIG. 1 shows a depiction of a silicon filter that may be microfabricated using photolithographic and chemical etching techniques. In this image, the 0.5 millimeter thick Si wafer is first processed to form a pattern of 450 μm deep wells. Then, smaller pores are patterned and etched through the remaining 50 μm thick Si at the bottom of each well. Two approaches to this second step are shown. Finally, the side of the membrane with small pores exposed (the bottom side in the image in FIG. 1) is coated with a reflective metal. This coating may be done using a variety of methods including chemical vapor deposition, thermal evaporation, sputtering, electrochemical or electroless deposition and other methods known to those skilled in the art of photolithographic/semiconductor processing. Another suitable coating or treating method includes chemical bonding by the use of reactive (e.g., photoreactive) chemical groups.

This is the side of the membrane onto which the microorganisms will be captured during filtration. The purpose of this reflective metal is to provide a high quality optical surface that has good reflection characteristics at the wavelengths that will be used for the optical inspection and spectroscopic detection methods described below. In this example, the flow path is directly through the pores, which are roughly perpendicular to the plane of the membrane. There are many other variations to the protocol discussed above that may be used to produce Si membranes having perpendicular pores. Those are included here by example.

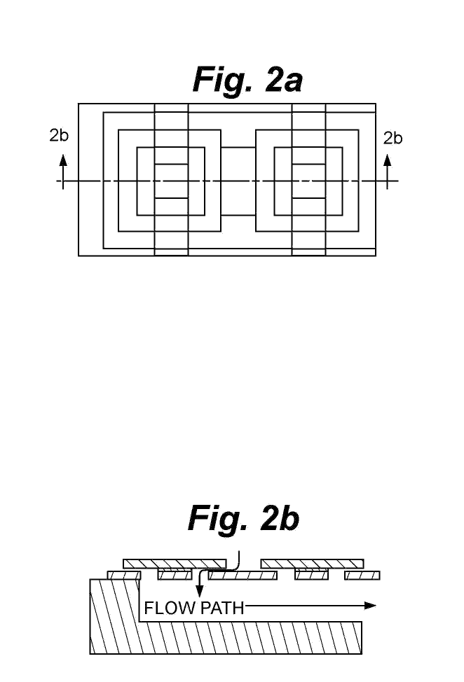

FIG. 2 shows another example of a Si filter made using standard microfabrication techniques, including sputtering, spin-coating of photoresist, chemical etching and other methods known to those skilled in the art of integrated circuit and microelectromechanical (MEM) device microfabrication. In this example, the top image is a top view (floorplan) of the basic membrane structure, and the bottom image is a side view (cross section) through the slice as indicated by the horizontal arrow in the top image. The flow path is shown by the arrows in the side view at the bottom of the figure. The size-selective filtration flow path is lateral, through the gap between the overlying green structure and the underlying red structure, and then out to the side. It also is possible to create holes through the gray base shown at the bottom of the side view image, such that the flow path can go through the gap between the green and red structures and then out through the holes in the gray structure and through the bottom of the filter membrane. Note that the green structures need not be squares or rectangles. They also may be round, oval, square, or other such shaped structures.

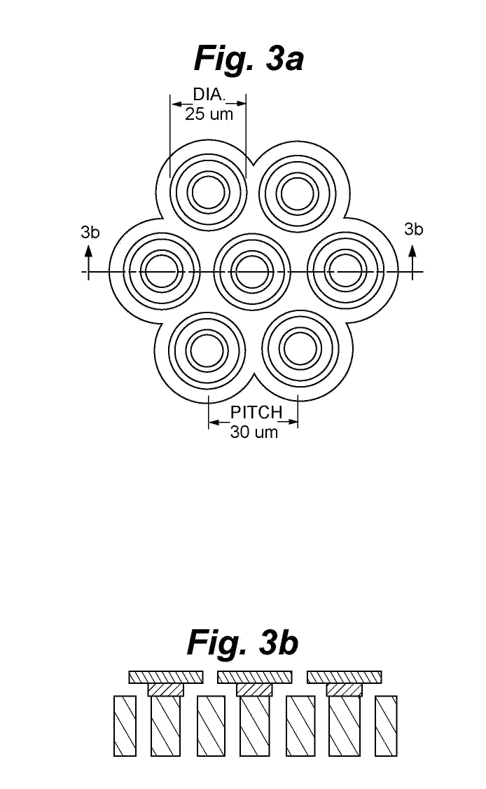

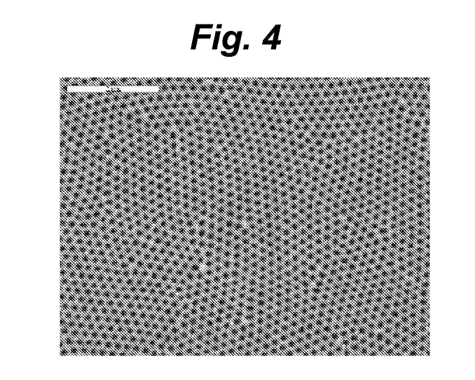

An example of a filter structure with rounded filtration structures is shown in FIG. 3. In this image the top view (top image) shows a hexagonal array of rounded filtration structures. This hexagonal array provides a high efficiency packing of the filtration structures and may provide better filtration rates. The cross section image at the bottom of FIG. 3 shows how the flow will occur. As in FIG. 2, fluid will flow between the blue top layer, through the gap between the blue and purple structures and out the bottom between the purple structures. As in FIG. 2, a base (i.e. the gray structure in FIG. 2) may be affixed to the bottom of the purple structures for structural support of the filtration structures. This base may have holes through it so fluid can flow out the bottom, or it may allow for fluid flow laterally, out the edges of the filter. Other filter designs based on this general concept of using fluid flow paths created using offset layered structures will be obvious to those skilled in the art of Si microfabrication and are included in the present invention. These offset layered structures may be comprised of various numbers of multiple layers (i.e. two or more). Such structures also are embodied in the present invention.

An advantage of such a round or oval shape is that there are no corners, and thus no structural variability associated with the sharpness of the corners. This may be important because such structural variability may make the optical inspection more difficult than it might be in the absence of such structural variability. In this example, the green, blue and red structures may be various solids, including but not limited to, metals, oxides, nitrides, Si, SiO₂ and polymers such as are used in integrated circuit microfabrication. Examples of such polymers include, but are not limited to, epoxy-based photoresist, such as SU-8, and polyimide photoresist, such as Kapton. Deposition of the green, blue and red structures may be done using any of a variety of methods known to those skilled in the art of integrated circuit and microelectromechanical (MEM) device microfabrication, such as vapor deposition, sputtering, electrochemical or electroless deposition, spin-coating, and the like.

There are several advantages of the types of membranes shown in FIGS. 1-3 above. First, because Si wafers have very flat surfaces, this approach may produce a filter that has a very flat and locally smooth surface. This flatness and smoothness have advantages in the optical and spectroscopic detection methods described below. Second, the use of an array of wells as in FIG. 1 endows the filtration membrane with more mechanical robustness than if only one large well were used. This is because the 0.5 mm thick regions between the wells provide structural support for the interior, etched region of the filter. This is especially important because of the vacuum manifolds typically used for filtering water samples. Such vacuums typically produce pressure differences across filtration membranes of approximately 50-100 kPa. Thus, the membrane must be designed to withstand such pressure differentials. A disadvantage of having unetched regions between the wells is that the total number of pores is reduced. This may cause the length of time required to filter a given sample volume with a given pressure differential to be longer. Thus, the use of a pattern of wells represents a trade-off between filtration rate and mechanical robustness. With this in mind, there are many modifications to this general type of design that may be used to optimize this trade off under a given set of operating conditions. For example, the width of the unetched portions may be increased. This will have the effect of increasing mechanical robustness but decreasing flow rate (because of a smaller number of pores) through the filter for a given pressure differential. Conversely, decreasing the width will reduce mechanical robustness while increasing flow rate. Such changes are obvious to those skilled in the art and encompassed by this description.

An additional advantage is that microfabrication processing produces multiple devices that are substantially the same and that have very low defect densities. This is important because defects may cause optical aberrations that interfere with the detection and identification of cells that grow into colonies. This lack of defects is not a characteristic of filters in widespread use for cell capture and detection.

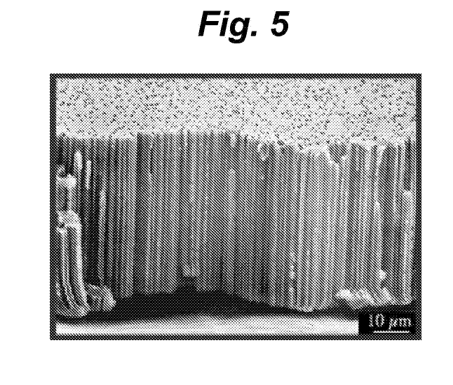

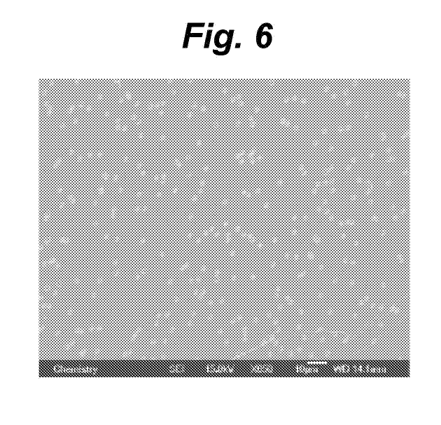

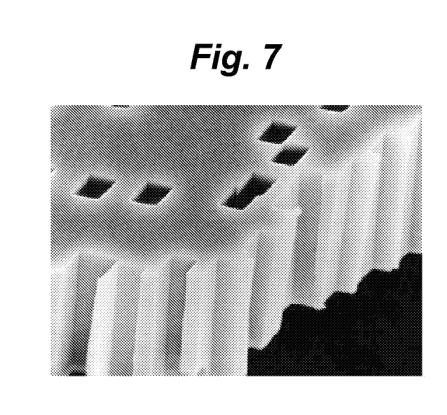

Alumina membranes also may be used for the filter material. FIG. 4 shows a top view of an alumina membrane. The white scale bar is one micron in length, which is on the order of the length of many microorganisms. FIG. 5 shows a cross-sectional view of a track-etched polycarbonate membrane. The top surface is seen to be quite flat, and the pores produced by the track-etching process are seen to pass directly and completely through the membrane. As part of the preparation of such track-etched membranes, it is possible to control the density of pores and the diameter of the pores. Pore density, in turn, can be used to affect flow rate and other useful characteristics of a membrane of this invention. Under some conditions it is possible to control the placement of the pores, for example by using a tracking source that can be steered (such as a high energy electron beam). FIG. 6 shows a top view of a track-etched mica membrane. This image shows how the placement of pores is random, which is caused by the radiation source that produces the tracks in the membrane. It also illustrates how smooth and flat the membrane surface is. In this image, the pore is approximately 0.5 micron across. This image also shows the extremely flat nature of the mica surface. FIG. 7 shows a close up of a mica membrane revealing that the tracks etched under some conditions are square, with very well-defined pores.

These membranes can also be coated (e.g., with thin films of various materials that endow the membrane with useful physical-chemical (e.g., optical) characteristics. Such characteristics can include altered or improved optical properties, such as reflectivity, and functional properties, such as the ability to alter the contact angle with water, thereby altering (and preferably enhancing) the flow rate, and other performance properties, such as desired interactions (e.g., binding) with cells themselves.

For example, they can be coated with thin films of gold to make the membrane surface reflective. They might also be coated with any of a variety of other metals. If light is used as part of the detection process, and if this light is reflected off of the surface as part of the detection process, it will be important that the coating on the membrane surface be highly reflective in the spectral region used for the detection. For example, if infrared light is used as part of the detection, then a metal that is highly reflective in the infrared, such as gold, may be used. If the coating conditions are controlled properly, the coating can be done so the pores through the filter membrane remain open. Thus, the microorganisms can be captured by filtration on the Au-coated surface of the membrane, and then optical and/or spectroscopic measurements can be made on the microorganisms that are present on this reflective surface. Alternatively, measurements can be made by transmitting light through the membrane.

There are many methods that may be used to coat the surfaces of these membranes, as described above. In a preferred embodiment, sputtering is used for the coating. This has the advantage of producing a thin (e.g., about one to about five nanometer thickness), substantially continuous metal film with optical properties that are substantially similar to those of bulk metal. It may be desirable to use sputter coating when coating metals that offer poor adhesion to surfaces such as mica, alumina, Si, SiO₂ or various polymers (e.g. epoxy-based, polyimide-based, etc.) used in photolithographic processing. This is because the sputtering process cleans the surface prior to deposition, thereby providing better adhesion than in many other metal deposition processes such as, thermal vapor deposition and electroless deposition.

Optionally, the filter material, including any portion or portions thereof (such as its surface) can be treated in order to alter or control various physical-chemical properties, including flow rates, interactions with cells or particulate matter, and so forth. Such treatments include, for instance, the use of thiol derivatives that can be immobilized on the gold coating on the filter to control the surface tension of the water sample at the surface, thereby allowing for control of the flow rate of the sample through the filter.

For instance, an additional feature of the metal used to coat the filters is that it permits one to take advantage of the surface chemistry of such metals to manipulate the flow of fluids through the filter. For example, for filters with small apertures, the surface tension of water may impede flow of the sample through the filter. If the wettability of the surface can be controlled through the immobilization of chemical compounds on the surface of the metal, then the flow rate may be increased. An example of this approach would involve the use of Au as the metal coating the filter membrane surface and a thiol compound as the immobilized chemical compound. Specifically, a compound such as HS(CH₂)₆OH may be easily immobilized onto the Au surface simply by immersion of the filter into an ethanol solution of the compound for a specified time. This time may range from a few minutes to as long as a day, depending on the degree of coverage desired for the compound on the surface. The immobilization proceeds by attachment of the SH group to the Au surface, resulting in loss of the H and formation of a Au—S bond that is quite strong. A result of this attachment is that the OH groups become pendant from the surface. Since these groups are quite hydrophilic, this renders the surface highly wettable toward water. Thus, this process makes the flow rate of the aqueous sample through the Au-coated filter membrane much faster than it would otherwise be with a simple Au coating. Because the time required for analysis is an important advantage of the present method, this surface treatment provides significant advantages. In a preferred embodiment, the metal coated filters have been treated with chemical compounds that increase the wettability of the filter structure toward water and aqueous solutions such that fluid flow rates during filtration are increased.

The preferred system further includes a growth medium adapted to permit the incubation and growth of microbes that may be retained on the filter surface. Once the sample has been captured on the filter, it can be placed on a growth medium. The purpose of this medium is to provide an environment in which any microorganisms captured on the surface of the membrane may be able to grow and reproduce. As described below, optical inspection can be used to monitor this growth, even at the level of single cells multiplying. Thus, cell growth provides a direct measure of viability, which is a critical issue in the determination of microorganism contamination in the various types of ultrapure water discussed above. Growth media provide essential nutrients for bacterial, yeast and fungal growth. Those basic essential components typically consist of a carbon source, a nitrogenous source, water and a component to maintain osmotic balance in the cell. A gelling agent can also be added to make the medium into a more solid-like material. Additional components may also be added to make the media more selective for a certain type of organism or to help differentiate organisms growing on the same medium.

Media can be prepared and used in various forms or states, e.g., as liquid, semi-solid and/or solid media. The first is commonly referred to as a broth, while the latter is commonly referred to as semi-solid agar or agar. The filter may be used in conjunction with any of the three media types listed above but for ease of discussion agar media will be used from this point forward. It is important that the filter be in physical contact with the medium to enable nutrients to diffuse through the filter. This will typically be accomplished by placing one side of the membrane filter directly onto the medium. These nutrients are then available for the bacteria to utilize for growth. Given the manner and extent to which nutrient must be provided through the pores, those skilled in the art will be able to select and control various parameters, such as the volume and/or viscosity of the growth medium. If the medium is has too low a viscosity, for instance, then it may move rapidly through the pores, carrying the cells away from the surface as it exits the pores. This will make the optical inspection impossible. It is too viscous, then nutrient flow through the pores may not be sufficient to allow for cell growth. Thus, proper control of the physical properties of the growth medium can contribute to the success of the measurement. In a preferred embodiment, the growth medium is a semi-solid or solid agar type of medium with flow characteristics that achieve the goals described above.

As described herein, and will become apparent to those skilled in the art, there are many types of media that may be used in conjunction with the current method for detection of viable microorganisms. One preferred media comprises a nutrient media that contain a carbon source, water, various salts, amino acids and nitrogen. (e.g., Tryptic Soy Agar (TSA), Nutrient Agar, Plate Count Agar (PCA), each of which are known to those skilled in the art of growth of microorganisms. A second type is minimal media, which contains similar components to the nutrient media but typically lacks the amino acids. (e.g., R2A agar and HPC Agar). Minimal media can be useful to recover stressed or viable but not culturable cells that can be found in other than a natural environment. This is especially relevant to the detection of viability for cells that are stressed by virtue of exposure to low nutrient conditions and/or high temperature such as might be found in a system that is being operated to produce PW or WFI.

A third type is selective media, which contain components that select for certain microorganisms. (e.g., m Endo Agar, XLD Agar, Eosin Methylene Blue Agar (EMB), Yeast and Mold (YM) media). These media provide certain nutrients that may select the growth of one type of microorganism and suppress the growth of another type of microorganisms. For example, it is possible to favor the growth of gram negative bacteria over gram positive bacteria by the addition of bile salts and/or crystal violet to the medium. A fourth type is differential media, which distinguish between organisms that may grow on the same medium. These media contain reagents that may produce changes in the appearance of the medium surrounding a colony of a particular type of microorganism compared to others. Such changes in appearance may typically be color changes. For example, Nutrient Agar with MUG (4-methylumbelliferyl-β-D-glucuronide) differentiates organisms that produce the enzyme glucuronidase (typically E. coli organisms) from other organisms. This test is typically used to distinguish fecal coliforms from total coliforms in a determination of water quality. Examples of differential media include MacConkey Agar, Eosin Methylene Blue (EMB) Agar, FC Agar. Additional types of media may be used and known to those skilled in the art. Temperature, humidity and oxygen content of the atmosphere also may impact bacterial growth, so these physical conditions may also be manipulated in such a way as to promote growth and/or favor growth of specific types of microorganisms.

In one embodiment, the filters with any captured microorganisms on their surface are placed onto a growth medium with characteristics such that nutrients can flow through the pores or other open structures in the membrane, thereby providing the possibility of growth of the microorganisms. In another embodiment, the growth medium may contain various combinations of components such that stressed microorganisms may grow successfully in a reasonable timeframe. In another embodiment, the growth medium may contain various combinations of components such that growth of specific types of microorganisms may be favored.

The system further includes an analytic device, e.g., microscope, adapted to permit analysis of the filter surface, within a predetermined incubation period, in order to determine whether the growth onset of viable microbes that may be present on the surface has begun.

Once the microorganisms are captured on a filter, the next step in the method is to determine whether or not the cells are viable, i.e. whether they will grow and reproduce. As discussed herein, this will typically involve placing the filter on a growth medium to supply the nutrients needed for growth of the cells.

In turn, this will typically involve the preparation and use of means to detect the growth. In one particularly preferred embodiment, cell growth, including the onset of cell growth, can be detected at the single cell level using a specific type of microscopy referred to as darkfield microscopy. This is a type of microscopy in which the illumination is supplied from the edge of the sample rather than the more traditional methods of epi-illumination (e.g., the supply of illumination in a reflectance geometry with illumination perpendicular to the sample plane) or transmission illumination (e.g., the supply of illumination from the far field beyond the sample).

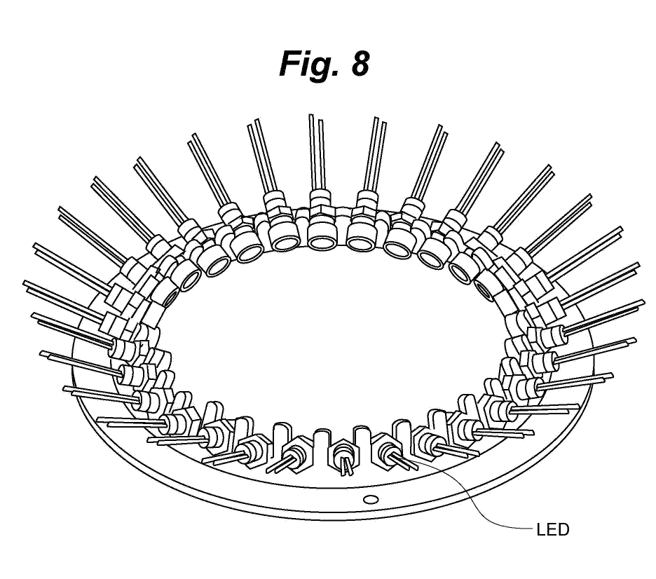

Modern microscopes typically use darkfield illumination in which the light travels through the sample support material such as a glass slide before striking the bacteria for what amounts to a light scattering observation. However, when using filters placed onto a growth medium, this normal darkfield geometry is not suitable since light will not propagate properly through the growth medium or the filter. In a preferred method as described here, the light is brought in by direct illumination (e.g., through space rather than through the sample support material). This geometry is schematically described in FIG. 8. In this geometry, light that simply strikes the filter surface will be reflected away. This light will not be collected through the microscope objective, and thus will not be detectable using the microscope. In contrast, light that strikes an object protruding from the surface, such as a microorganism captured on the surface of the filter, will be scattered. This light will be collected by the microscope objective and may be detected by the microscope. If a digital camera or other type of imaging detector is used, one will observe a bright spot for the protruding object imaged against a darker background. This allows one to locate microorganisms on the filter surface. Thus, in this way, it is possible to detect the location, shape and size of microorganisms on the surface of the surface of the filter.

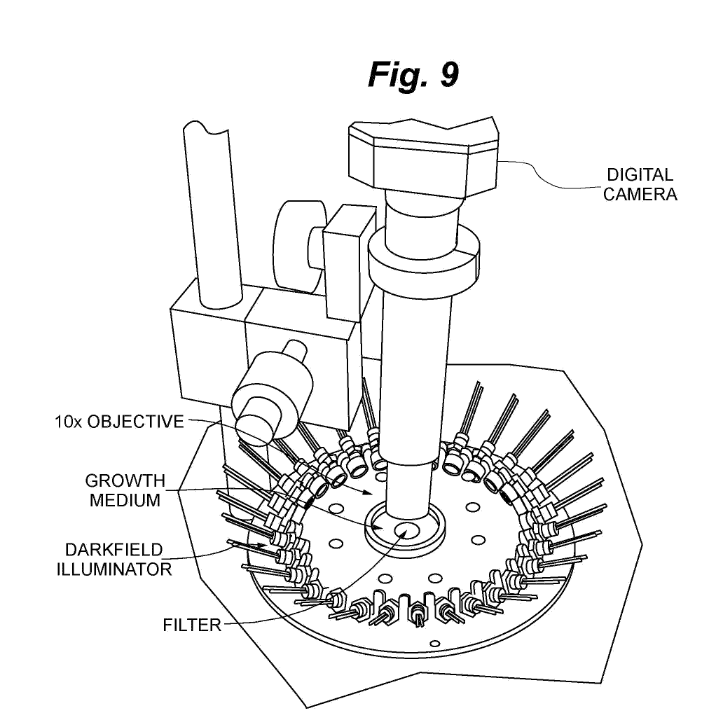

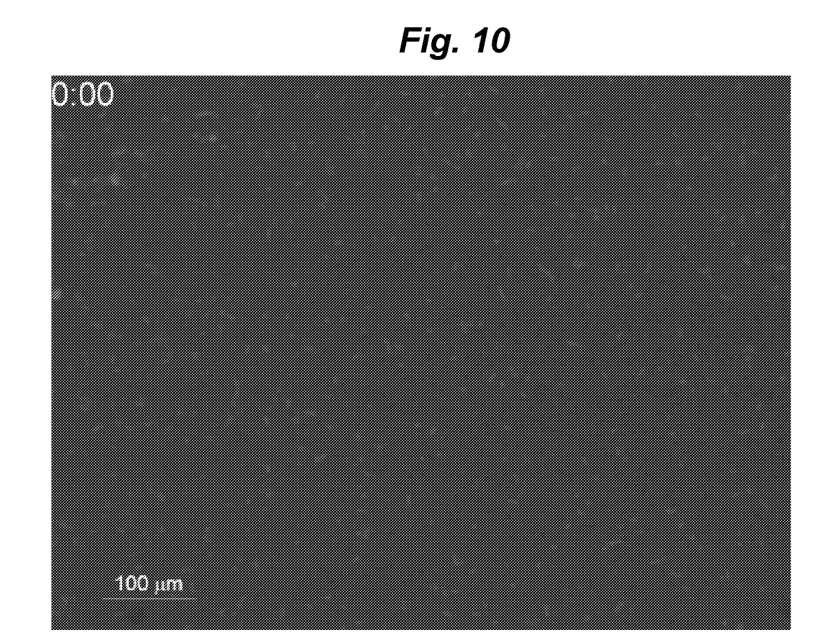

FIG. 8 shows a schematic of the darkfield illuminator used for monitoring growth of microorganisms in the present invention. The ring contains multiple LEDs (light emitted diodes) situated 360° around the sample with the LEDs pointed directly toward the center of the sample stage. In one embodiment, one could use LEDs with specific wavelengths to enhance the contrast of the image. In another embodiment, white light output can be used to allow observation of color changes during the colony formation, such as might occur when using differential media as described above. FIG. 9 shows a picture of the actual experimental apparatus used for the darkfield images described below. The picture shows a 10× objective used for the measurement. However, as known to those skilled in the art, different magnifications may be used for such measurements, depending on the type of sample being imaged, the optical resolution desired and the field of view desired. The figure also shows a digital camera mounted on top of the microscope. This camera is used to obtain images of the surface, and also to determine the color of the objects identified in the image (see Example 2 below).

In turn, the system can provide either qualitative and/or quantitative results regarding the onset of microbial growth, and in turn, can be used to determine and/or distinguish as between the existence of a) particulate matter in the sample, b) living cells that are not culturable under the conditions of use, and/or c) cells that are both living and culturable, as evidenced by their ability to exhibit the onset of detectable growth on the surface within the predetermined incubation period. Qualitative results can include, for instance, the determination of whether microbes are present at all in a sample, or at a level above a predetermined threshold (that is, semi-quantitative). Quantitative results can include the determination of actual and/or relative cell types and numbers, as between the various microbes originally present in the sample.

Typically, the system will be used to distinguish living, culturable cells from any other materials (e.g., particulate matter together with non-culturable cells) that may be present in the sample. In turn, the present invention provides various combinations and subcombinations of components, which have the potential to be novel in their own right, including filter materials and/or growth media adapted for use in a method as described herein. The invention further provides, for instance, a method for the evaluation of a liquid sample such as water, by use of a system as described herein, as well as a liquid sample, per se, which has been sampled for microbes according to a method and/or using a system as described herein.

### EXAMPLES

### Example 1

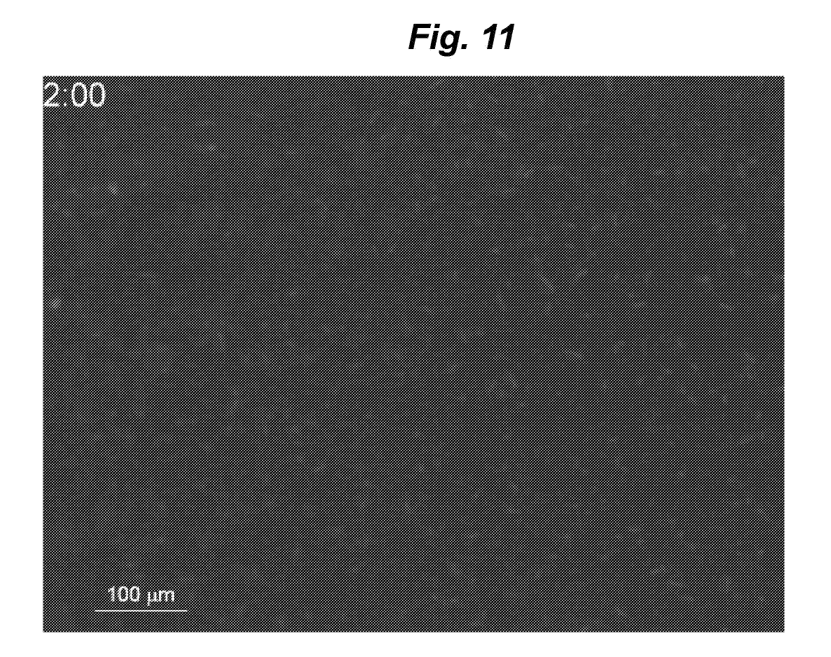

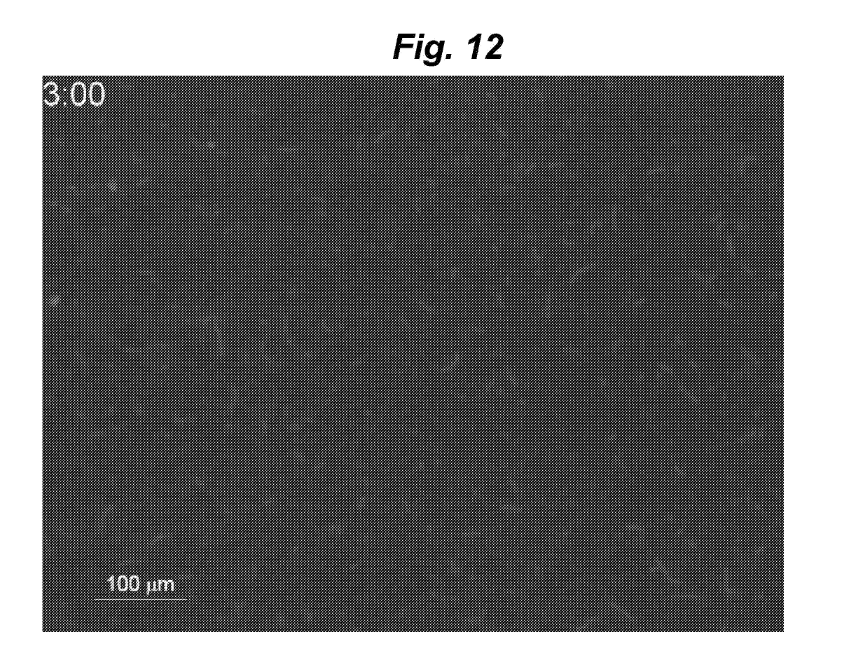

A solution containing Bacillus subtilis was filtered through a gold coated track-etched polycarbonate filter with 0.4 μm pore size. After the filtration, the filter was positioned on a Petri dish filled with growth medium. A microscope equipped with a digital camera was used to image as area of the filter with approximate dimensions of 800 μm×600 μm. Then, this same area of the filter was monitored over a time frame of three hours. FIG. 10 shows the darkfield image obtained immediately after the filtration. The locations of bacterial cells can be seen as brighter objects against the darker background. As can be seen, some objects appear to be single cells, while some appear to be several cells aggregated together. FIGS. 11 and 12 show the same location on the filter after two and three hours, respectively, of exposure to the growth medium. Close inspection reveals that many of the cells have grown and reproduced during the exposure time, as evidenced by the increase in size and the elongation in the shape of many of the objects. Thus, the darkfield illumination provides a simple means of detecting viability of the microorganisms imaged on the filter. In addition, one could easily accelerate the growth of the bacteria by raising environmental temperature to 37° C., thereby providing an even more rapid method of detecting viability.

### Example 2

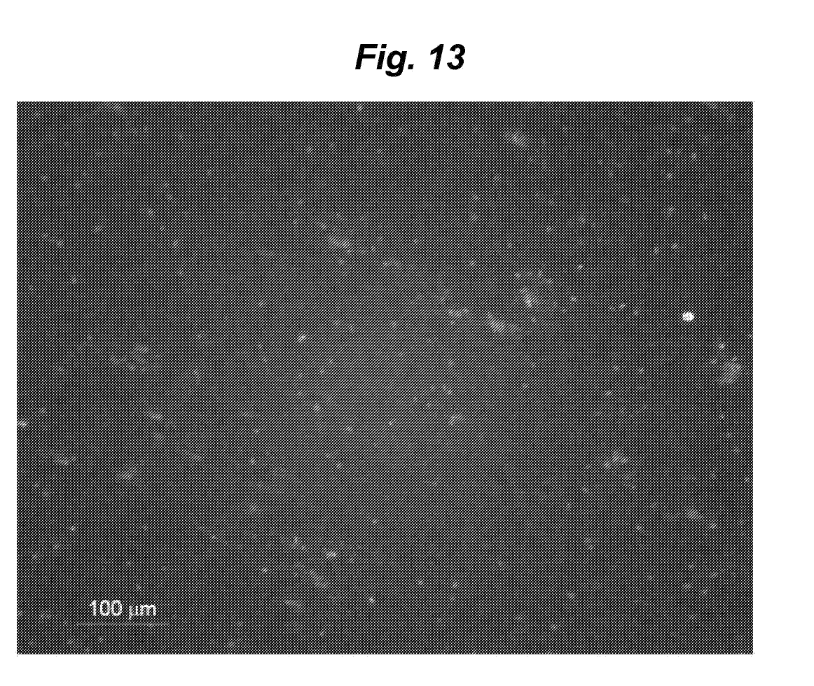

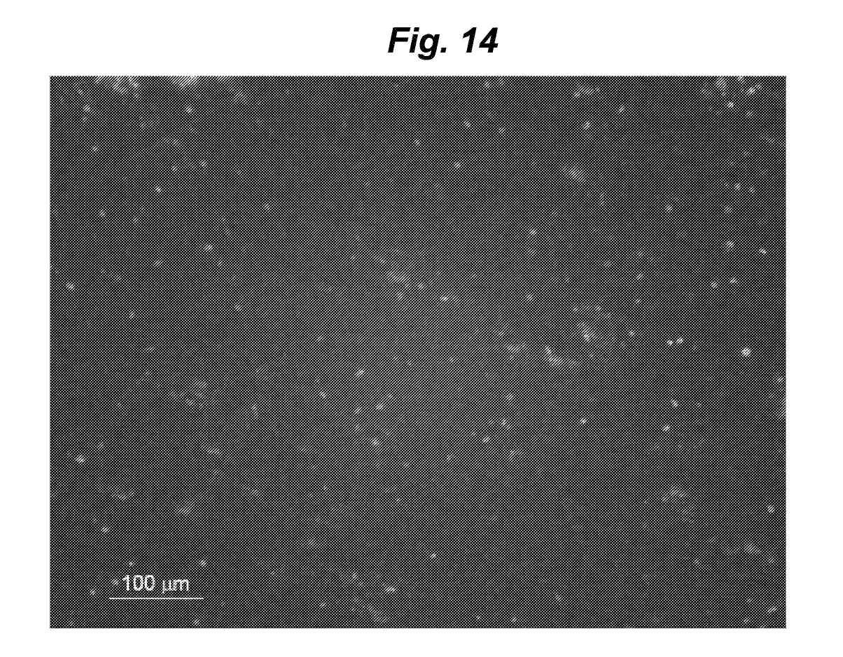

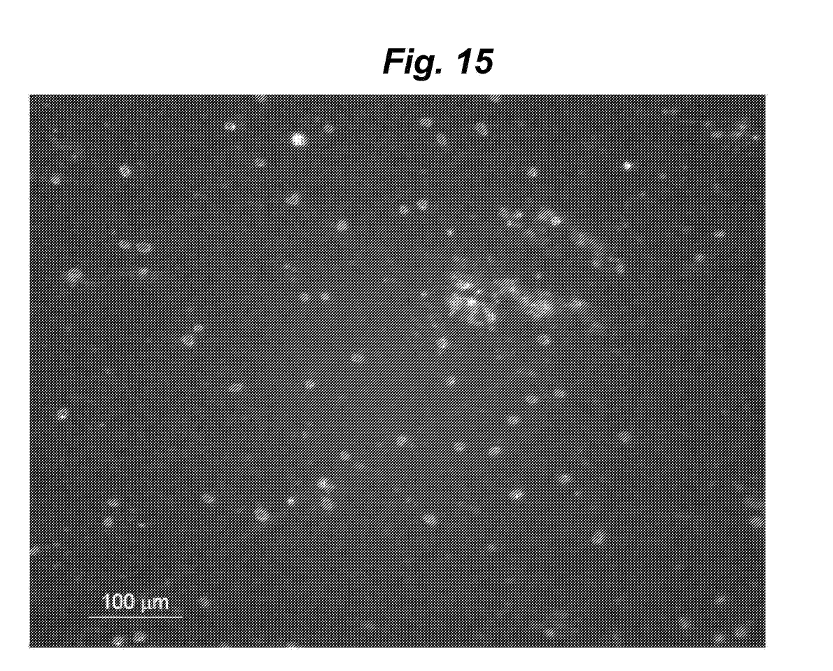

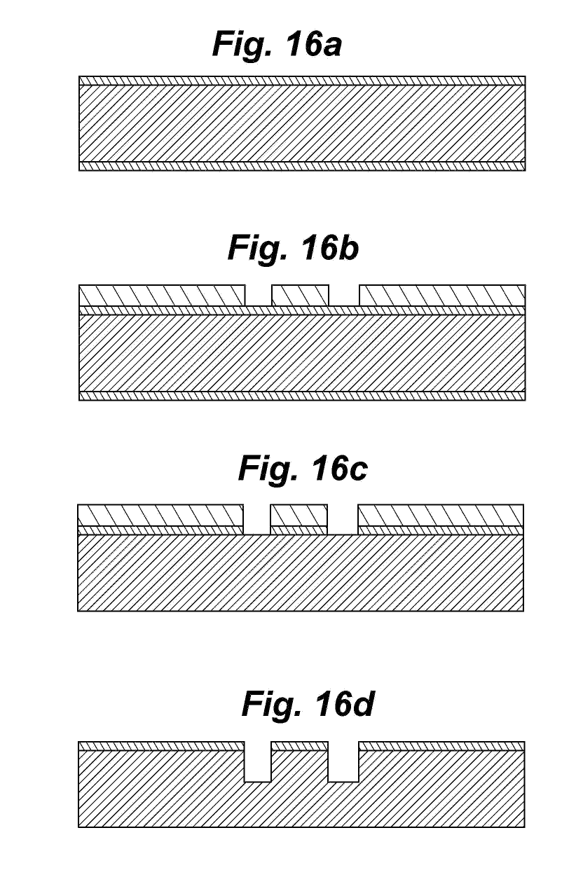

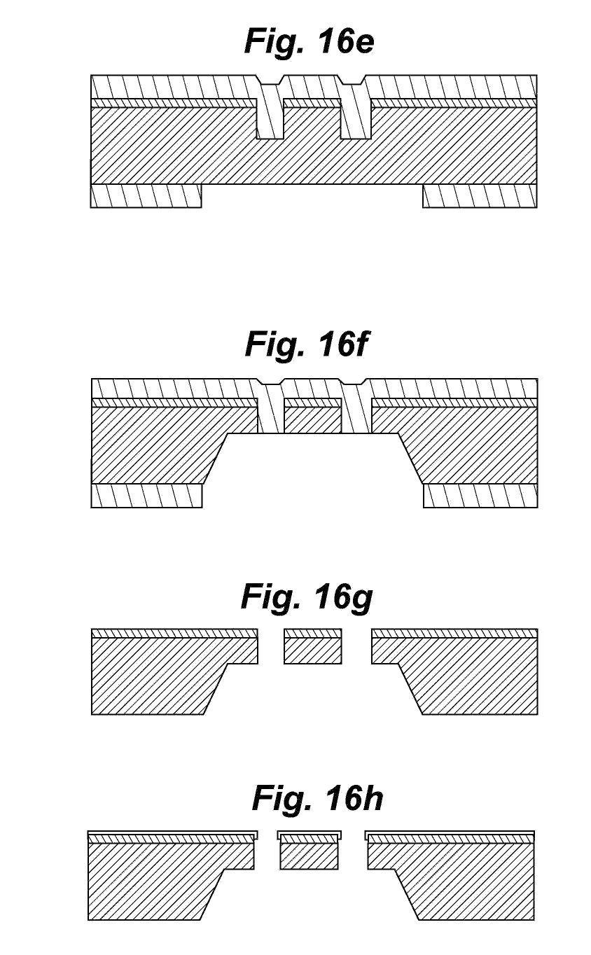

A solution containing E. coli was filtered through the same type of filter mentioned in Example 1. Instead of positioning the filter after filtration on to a universal growth medium, the filter was positioned on Endo agar. Endo type agars (such as m Endo agar) can provide a selective culture medium for coliforms and other enteric microorganisms that will inhibit gram-positive microorganism growth. E. coli and coliform bacteria metabolize lactose in the Endo medium with the production of aldehyde and acid. The aldehyde liberates fuchsin from a fuchsin-sulfite compound present in the medium and turns the coliform colonies red with a golden metallic sheen. In this example, the environmental temperature was kept at 37° C. for rapid bacterial growth. FIG. 13 shows the initial darkfield image after filtration. The bright spots indicate the location of various objects on the filter surface. These objects could be either E. coli or particles. FIG. 14 shows the same filter location after 50 minutes. As can be seen, several objects have increased in size during the 50 minute period, indicating that those objects were microorganisms that grew during the 50 minute period. FIG. 15 shows the same area of the filter after two hours of growth. Again, one can clearly see the continued growth of several of the objects initially observed on the filter surface. In addition, one can clearly observe the reddish color of the spots after continued growth. The change of color indicates the presence of E. coli or other coliform microorganisms. When observing light scattering using darkfield illumination, the light scattering phenomenon creates a white shape surrounding the microorganisms or other particulate objects as shown in FIG. 15. The light may also penetrate into the microorganisms since the cell wall is not opaque. The metallic red color that is formed in the microcolonies is the result of a blue absorbing dye that is produced as part of the reaction in the differential medium. In this case, the light scattering that occurs is dominated by red scattering, since the blue light is absorbed in the microcolony. Thus, the microcolonies appear red.

These examples show the manner in which samples can be filtered to capture microorganisms and then optically inspected using darkfield microscopy under growth conditions to determine viability. It is also possible to use software to examine each object in the darkfield image individually so as to observe any changes in its size or shape during the growth period. This is easily accomplished using any of a variety of pattern recognition or image processing algorithms.

We claim:

1. A system for the rapid detection of microbial contamination in a fluid sample such as water, the system comprising:
a) a filter assembly comprising a filter material comprising a surface adapted to receive the sample in order to retain microbes from the sample, and preferably substantially all microbes, on the filter surface under conditions that minimize the potential for contamination from sources other than the sample itself, and in a manner that permits the filter surface to itself then be incubated in order to permit the growth onset of viable microbes contained thereon;
b) a growth medium adapted to permit the incubation and growth of microbes that may be retained on the filter surface, and
c) an analytic instrument adapted to permit analysis of the filter surface, within a predetermined incubation period, in order to determine whether and/or the extent to which the growth onset of viable microbes that may be present on the surface has begun;
whereby, the system can provide either qualitative and/or quantitative results regarding the onset of microbial growth, and in turn, can be used to determine and/or distinguish as between the existence of a) particulate matter in the sample, b) living cells that are not culturable under the conditions of use, and/or c) cells that are both living and culturable, as evidenced by their ability to exhibit the onset of detectable growth on the surface within the predetermined incubation period.

2. A system according to claim 1 wherein the filter material is optically transparent and substantially free of optical imperfections.

3. A system according to claim 2 wherein the filter material is microfabricated from mica, silicon, aluminum oxide membranes, cellophane or cellulose-based membranes, polymeric materials selected from the group consisting of polycarbonate, and polyimide, and membranes formed from combinations thereof.

4. A system according to claim 3, wherein the filter material provides pores having suitable shape, size, and direction to permit the flow of fluid therethrough in a manner that retains substantially all microbes on the surface thereof.

5. A system according to claim 4 wherein the pore direction is selected from perpendicular and parallel to the filter surface, and combinations thereof, and the pore shape is selected from circular, oval and square, and combinations thereof.

6. A system according to claim 5 wherein the pores are prepared in the filter material by a method selected from the group consisting of: photolithographic methods, chemical etching, punching holes mechanically, ablating holes, and forming holes in the membrane by use of a mold.

7. A system according to claim 6 wherein the surface is coated or treated to improve its physical-chemical properties.

8. A system according to claim 7 wherein coating or treating is selected from the group consisting of sputtering, electrochemical or electroless deposition, spin-coating, chemical vapor deposition, thermal evaporation, and chemical bonding by use of reactive groups.

9. A system according to claim 7 wherein the coating or treatment is used to alter or improve properties selected from the group consisting of optical properties, flow rates, and cell interactions.

10. The system of claim 1, wherein the sample is selected from gaseous and liquid samples.

11. The system of claim 1 wherein a sample protocol is used to obtain a sample from a liquid that is expected to have, at most, a very low level of microbial contamination, the protocol comprising the use of aseptic sampling.

12. The system of claim 1 wherein the filter material provides a filter surface that is adapted to retain any microbes that may be present in the sample, on the surface of the filter itself, such that they can be grown and detected using darkfield microscopy.

13. The system of claim 1 wherein the growth onset of viable microbes can be determined within on the order of eight hours or less incubation on the filter surface.

14. The system of claim 1 wherein the instrument is a microscope and further comprises a darkfield microscope.

15. A method of sampling a liquid, comprising the steps of providing a system according to claim 1, and employing the system to sample the liquid and to determine the presence of microbial growth on the surface of the membrane surface.

16. A filter assembly for use in a system of claim 1, wherein the filter assembly comprises a filter material suitable to filter the sample in order to retain microbes present therein, to then retain the filter material in the course of incubation and growth of microbes that may be retained on the filter surface, and to then be operably coupled with the analytic instrument to permit analysis of the filter surface.

17. A filter material for use in a system of claim 1, comprising a filter material that has been microfabricated to permit the flow of a sample therethrough, in a manner that permits substantially all microbes contained in the sample to be retained on the surface thereof.

18. An analytic instrument for use in a system of claim 1, the instrument being adapted to retain the filter assembly and/or filter material, in the course of analyzing the filter surface in order to determine whether and/or the extent to which the growth onset of viable microbes that may be present on the surface has begun.
